(19) United States
(12) Patent Application Publication
(10) Pub. No.: US 2005/0228495 A1
(43) Pub. Date: Oct. 13, 2005

(54) Suspended heart valve devices, systems, and methods for supplementing, repairing, or replacing a native heart valve

(76) Inventor: John A. Macoviak, La Jolla, CA (US)

Correspondence Address: RYAN KROMHOLZ & MANION, S.C., POST OFFICE BOX 26618, MILWAUKEE, WI, 53226, US

(21) Appl. No.: 11/036,745

(22) Filed: Jan. 14, 2005

Related U.S. Application Data
(60) Provisional application No. 60/536,601, filed on Jan. 15, 2004.

(51) Int. Cl. A61F 2/24

(52) U.S. Cl. 623/2.18

(57) ABSTRACT

A valve prosthesis is sized and configured to rest within a blood path subject to antegrade and retrograde blood flow. A trestle element on the prosthesis extends across the blood path. A leaflet assembly is suspended from the trestle element and extends into the blood path in alignment with blood flow. At least one mobile leaflet member on the leaflet assembly is sized and configured to assume orientations that change according to blood flow direction. The mobile leaflet member has a first orientation that permits antegrade blood flow and a second orientation that resists retrograde blood flow. The valve prosthesis, when implanted in a heart chamber or great vessel, serves to supplement and/or repair and/or replace native one-way heart valve function.

### RELATED APPLICATION

This application claims the benefit of U.S. Provisional Patent Application Ser. No. 60/536,601, filed 15 Jan. 2004, and entitled “Suspended Heart Valve Devices, Systems, and Methods for Supplementing, Repairing or Replacing a Native Heart Valve.”

### FIELD OF THE INVENTION

The invention is directed to devices, systems, and methods for improving the function of a native heart valve.

### BACKGROUND OF THE INVENTION

The heart (see FIG. 1) is a double (left and right side), self-adjusting muscular pump, the parts of which work in unison to propel blood to all parts of the body. The right side of the heart receives poorly oxygenated (“venous”) blood from the body from the superior vena cava and inferior vena cava and pumps it through the pulmonary artery to the lungs for oxygenation. The left side receives well-oxygenation (“arterial”) blood from the lungs through the pulmonary veins and pumps it into the aorta for distribution to the body.

The heart has four chambers, two on each side—the right and left atria, and the right and left ventricles. The atria are the blood-receiving chambers, which pump blood into the ventricles. A wall composed of membranous and muscular parts, called the interatrial septum, separates the right and left atria. The ventricles are the blood-discharging chambers. A wall composed of membranous and muscular parts, called the interventricular septum, separates the right and left ventricles.

The synchronous pumping actions of the left and right sides of the heart constitute the cardiac cycle. The cycle begins with a period of ventricular relaxation, called ventricular diastole. The cycle ends with a period of ventricular contraction, called ventricular systole.

The heart has four valves (see FIGS. 2 and 3) that ensure that blood does not flow in the wrong direction during the cardiac cycle; that is, to ensure that the blood does not back flow from the ventricles into the corresponding atria, or back flow from the arteries into the corresponding ventricles. The valve between the left atrium and the left ventricle is the mitral valve. The valve between the right atrium and the right ventricle is the tricuspid valve. The pulmonary valve is at the opening of the pulmonary artery. The aortic valve is at the opening of the aorta.

At the beginning of ventricular diastole (i.e., ventricular filling)(see FIG. 2), the aortic and pulmonary valves are closed to prevent back flow from the arteries into the ventricles. Shortly thereafter, the tricuspid and mitral valves open (as FIG. 2 shows), to allow flow from the atria into the corresponding ventricles. Shortly after ventricular systole (i.e., ventricular emptying) begins, the tricuspid and mitral valves close (see FIG. 3)—to prevent back flow from the ventricles into the corresponding atria—and the aortic and pulmonary valves open—to permit discharge of blood into the arteries from the corresponding ventricles.

Heart valves have mutually coapting leaflets. The mitral valve has two mutually coapting leaflets, and the tricuspid, pulmonary, and aortic valves each have three mutually coapting leaflets. In all heart valves, the outside edge or base of each leaflet is joined to the valve annulus The valve annulus comprises a fibrous ring of collagen that forms a part of the fibrous skeleton of the heart. In all heart valves, the inside edge of each leaflet occupies the lumen of the valve. All inside leaflet edges are free of contact with the annulus and, in a healthy heart, coapted with each other at or near the middle region of the valve lumen.

The leaflets receive chordae tendinae (cords) from papillary muscles. In a healthy heart, these muscles and their tendinous cords support the valves. The peripheral attachment of the outer edges of the leaflets to the native valve annulus serves as a hinge, allowing swinging movement of the leaflets between opened and closed positions in response to hemodynamic forces in the heart.

For example, the aortic valve opens by hemodynamic forces being exerted on the upstream or inferior surface of the leaflets, due to contraction of the left ventricle. The leaflets swing open toward the periphery of the valve annulus, to permit blood flow out of the left ventricle and into the aorta. When left ventricular contraction ceases, blood downstream to the valve (i.e., in the aorta) rushes back toward the valve. The valve closes to prevent retrograde blood flow into the left ventricle. Closure of the leaflets occurs when blood on the downstream or superior surface of the leaflets exerts a push from above, to cause each of the three, semi-lunar leaflets to form a one-third cup or cone. Compositely, the three semi-lunar leaflets coapt to form a full cup or cone. The attachment of the outer edges to the annulus, and the leaflet-to-leaflet coapting contact along the inner edges, buttress the coapting leaflets one against another. Since the leaflet is semi-lunar, the buttressing of adjacent leaflet prevents the individual leaflets from prolapsing, which would render the valve incompetent and result in regurgitant blood flow through the valve in the wrong direction from the aorta into the left ventricle.

Because of the nature of its structure and function, the aortic valve—like all native heart valves—can be classified as a “central flow” type of valve. That is, the flow path of blood through the valve, when the leaflets are opened, is generally through the center region of the valve. Because the outer edges of the leaflets are attached to the annulus, there is no blood flow in the peripheral regions of the valve.

The central flow characteristics of the native aortic valve has served as a model for conventional tissue type bioprosthetic heart valves. The leaflets of conventional bioprosthetic heart valves typically comprise animal tissues preserved with glutaraldehyde. These tissues include pericardium or xenograft aortic valve leaflets. The valve leaflets are all attached along their outside edges to a valve-housing and present semi-lunar shaped, free-edges that coapt among adjacent leaflets during valve closure. Generally, there are three such leaflets, which are unattached to anything else other than the valve housing along their outer edges. The free edge interactions of these usually semi-lunar shaped leaflets allow the leaflets to open away from the central orifice of the valve, with the leaflets being pushed out toward the periphery by the central flow of blood through the valve. Cyclically the valves close by the leaflets falling back centrally toward adjacent leaflets.

Due to their central flow characteristics, conventional bioprosthetic heart valves depend upon a relatively bulky, annulus-like structures for support and attachment of the leaflets. Such structures are required to impart to the leaflets the resistance necessary to prevent leaflet prolapse in the face of the high pressure developed during contractions (pumping) of the left or right ventricles. Such requirements are inherent in any central flow type prosthetic valve for use in the heart. These requirements limit the compressibility and flexibility of the valve, making intravascular deployment problematic, at best.

### SUMMARY OF THE INVENTION

The invention provides devices, systems and methods that supplement, repair, or replace a native heart valve. The devices, systems, and methods include a valve prosthesis that does not possess the characteristics of a central flow valve type. Instead, the valve prosthesis is sized and configured to serve as a peripheral flow suspension valve. The term “peripheral flow” denotes that, unlike a conventional central flow valve, the valve functions by allowing blood flow at the periphery of one or more mobile leaflets members, so that the flow lumen of the valve is outside all mobile leaflet members. Peripheral flow channels are located between a given mobile leaflet member and a mural wall of a heart, great vessel or native valve annulus. The term “suspension” denotes that, unlike a conventional central flow valve, the valve leaflets are suspended from a trestle above an annulus.

The unique design of a peripheral flow suspension valve better allows intra-vascular placement of a heart valve, due to its enhanced collapsibility. Unlike a central flow valve, a peripheral flow suspension valve does not require a substantial valve housing at its periphery for holding leaflets in place. A peripheral flow suspension valve makes possible a valve prosthesis having greater compressibility and flexibility relative to convention central flow valves.

Other features and advantages of the invention shall be apparent based upon the accompanying description, drawings, and claims.

### DESCRIPTION OF THE DRAWINGS

FIG. 1 is a perspective, anterior anatomic view of the interior of a healthy heart.

FIG. 2 is a superior anatomic view of the interior of a healthy heart, with the atria removed, showing the condition of the heart valves during ventricular diastole.

FIG. 3 is a superior anatomic view of the interior of a healthy heart, with the atria removed, showing the condition of the heart valves during ventricular systole.

FIGS. 4A and 4B are perspective views of a valve prosthesis comprising a frame base and a trestle that spans across and above the frame base to support a leaflet assembly comprising two mobile leaflet members that assume different complementing orientations in response to blood flow, FIG. 4A showing the mobile leaflet members in a complementing orientation allowing antegrade flow and FIG. 4B showing the mobile leaflet members in a different complementing orientation blocking retrograde flow.

FIGS. 5A and 5B are perspective views of another embodiment of a valve prosthesis comprising a frame base and a trestle that spans across and above the frame base to support a leaflet assembly comprising one mobile leaflet member and one immobile leaflet member, FIG. 5A showing the mobile and immobile leaflet members in a complementing orientation allowing antegrade flow and FIG. 5B showing the mobile and immobile leaflet members in a different complementing orientation blocking retrograde flow.

FIGS. 6A, 6B, and 6C are perspective views of another embodiment of a valve prosthesis comprising an interrupted frame base and a trestle component that spans across and above the frame base to support a leaflet assembly comprising two mobile leaflet members, FIG. 6A showing the valve prosthesis in an exploded view, FIG. 6B showing the valve prosthesis in one representative assembled view with an open-loop configuration, and FIG. 6C showing the valve prosthesis in another representative assembled view with a close-loop configuration.

FIGS. 7A and 7B are perspective views of another embodiment of a valve prosthesis comprising an sliding frame base and a trestle component that spans across and above the frame base to support a leaflet assembly comprising two mobile leaflet members, FIG. 7A showing the valve prosthesis in an exploded view, and FIG. 7B showing the valve prosthesis in one representative assembled view.

FIGS. 8A and 8B are perspective views of another embodiment of a valve prosthesis comprising an interrupted frame base and a trestle that spans across and above the frame base to support a leaflet assembly comprising one mobile leaflet member and one immobile leaflet member, FIG. 8A showing the valve prosthesis in one representative assembled view with an open-loop configuration, and FIG. 8B showing the valve prosthesis in another representative assembled view with a close-loop configuration.

FIGS. 9A, 9B, and 9C are perspective views of another embodiment of a valve prosthesis comprising a tripod-like trestle structure formed by three interlocking trestle members that support a leaflet assembly comprising permutations of mobile and immobile leaflet members, FIG. 9A showing in exploded view the trestle structure and an associated frame base, FIG. 9B showing the trestle structure and frame base in one representative assembled view, and FIG. 9C showing the trestle structure in another representative assembled view free of a frame base.

FIGS. 10A and 10B are perspective views of another embodiment of a valve prosthesis comprising a frame base and a trestle that spans across and above the frame base to support a leaflet assembly comprising one mobile leaflet member and one immobile leaflet member, the leaflet assembly including gaps or holes that allow blood to circulate through the interior of the leaflet assembly to perform a washing function, FIG. 10A showing the mobile and immobile leaflet members in a complementing orientation blocking retrograde flow with blood leakage through the gaps and FIG. 10B showing the mobile and immobile leaflet members in a different complementing orientation allowing antegrade flow.

FIGS. 11 and 12 are perspective, anterior anatomic views of the interior of a heart in which valve prostheses like that shown in FIGS. 4A and 4B have been implanted, one in the vicinity of the aortic valve and one in the vicinity of the mitral valve, FIG. 11 showing the functioning of the valve prostheses during ventricular diastole and FIG. 12 showing the functioning of the valve prostheses during ventricular systole.

FIGS. 13A, 13B, and 13C are perspective, anterior anatomic views of the interior of a heart showing the implantation of a valve prosthesis like that shown in FIGS. 4A and 4B by intra-vascular approach.

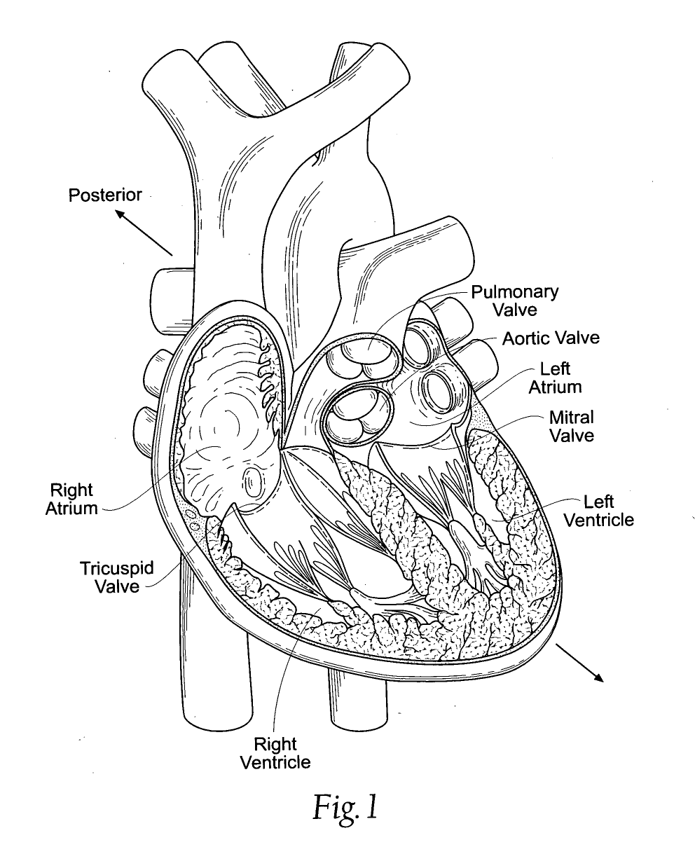

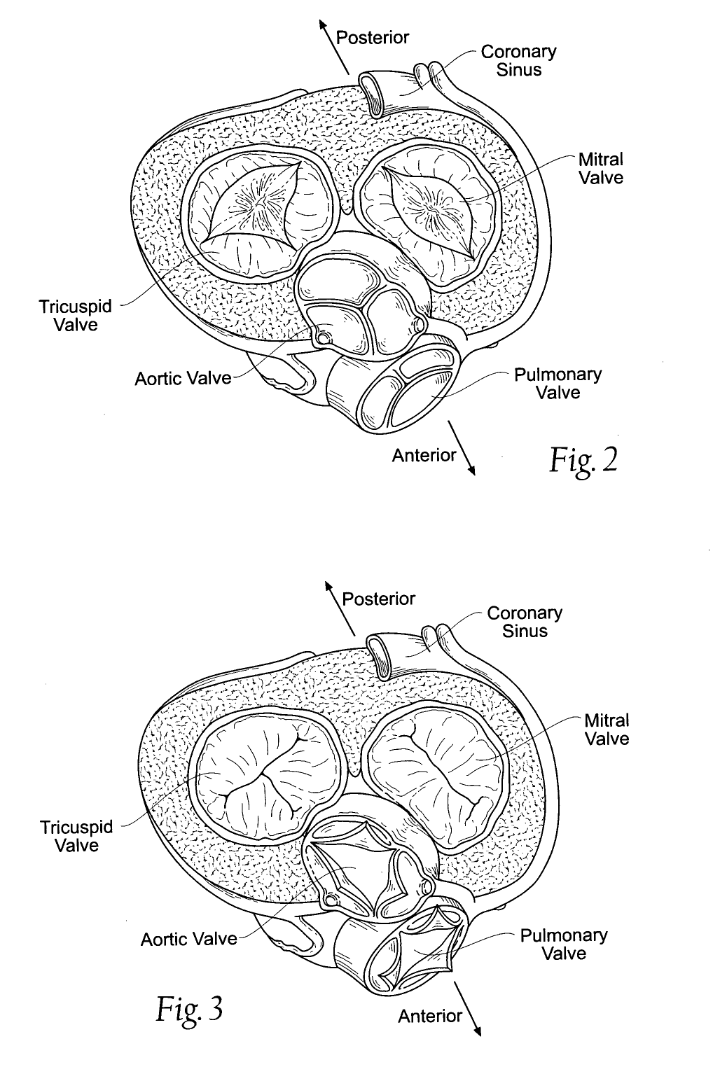

### DETAILED DESCRIPTION

Although the disclosure hereof is detailed and exact to enable those skilled in the art to practice the invention, the physical embodiments herein disclosed merely exemplify the invention, which may be embodied in other specific structure. While the preferred embodiment has been described, the details may be changed without departing from the invention, which is defined by the claims.

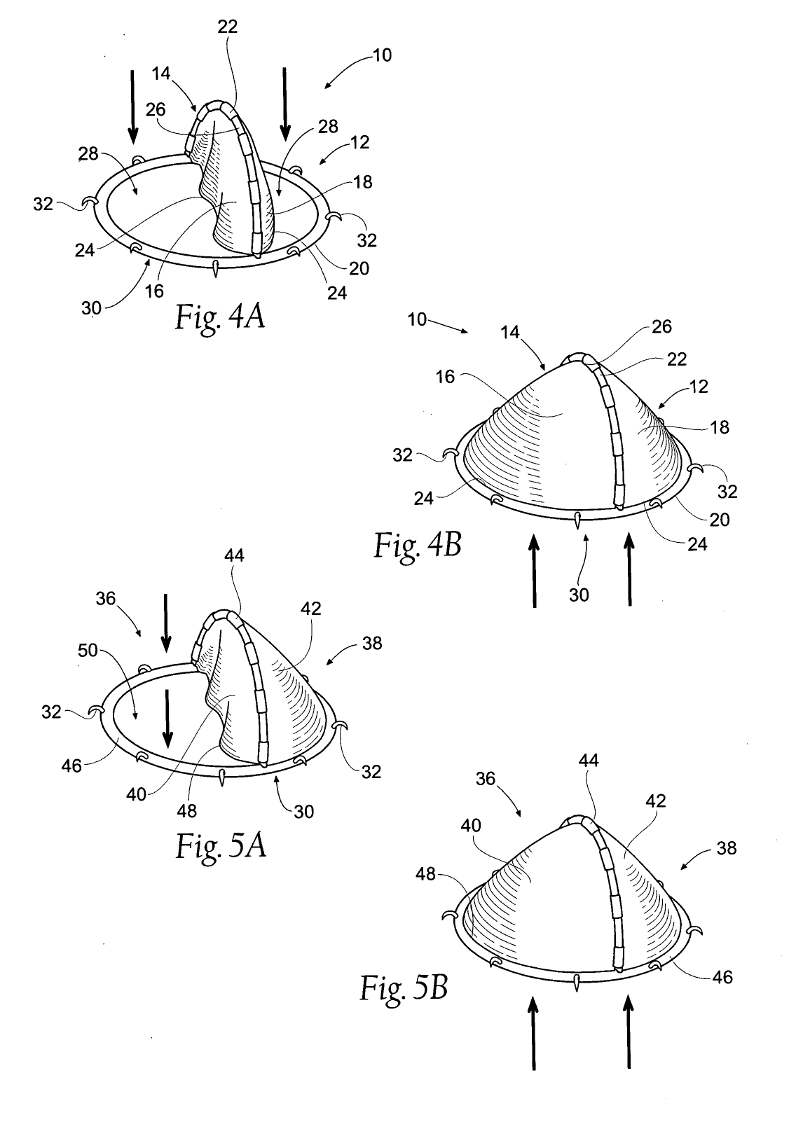

FIGS. 4A and 4B show one embodiment of a heart valve prosthesis 10 that embodies features of the invention. The heart valve prosthesis 10 is sized and configured to supplement, repair, or replace a native heart valve. In its most basic form, the prosthesis 10 comprises a skeleton or frame 12 that supports a leaflet assembly 14. The leaflet assembly 14 includes at least two leaflet members 16 and 18. At least one of the leaflet members is mobile. In the embodiment shown in FIGS. 4A and 4B, both leaflet members 16 and 18 are mobile.

The frame 12 may comprise an elastic or inelastic metal or polymeric material, like nitinol or malleable stainless steel. This construction enables intravascular implantation of the frame 12. For open surgical implantation, the frame 12 may comprise inelastic metal or polymeric composition. In surgical versions, the frame 12 may be more robust, with less concern of compressing the valve for trans-vascular delivery and implantation. Thus, in surgical version, more traditional inelastic materials like stainless steel rather than shaped memory alloys may be used.

In the embodiment shown in FIGS. 4A and 4B, the frame 12 comprises two basic structural components; namely, a frame base 20 and a leaflet support trestle 22.

The frame base 20 is sized and configured to engage a generally circular shape of a native valve annulus or great vessel lumen where it is intended to be implanted and dwell. The frame base 20 may be variously constructed. The frame base 20 can take various shapes and have various cross-sectional geometries. The frame base 20 can have, e.g., a generally curvilinear (i.e., round or oval) cross-section, or a generally rectilinear cross section (i.e., square or rectangular), or combinations thereof.

In FIGS. 4A and 4B, the frame base 20 takes the form of a continuous ring. Interrupted or sliding frame base structures can be used, as will be described in greater detail later. The frame base 20 may be made of spring-memory metal or polymer materials that make it self-expanding, or a malleable metal or polymer material that can be expanded in response to an external expansion force, e.g., a balloon.

The leaflet support trestle 22 spans across and above the central region of the frame base 20. The leaflet support trestle 22 is supported at its opposite ends by attachment to the frame base 20. The leaflet support trestle 22 may comprise an elastic or inelastic metal or polymeric material. Desirably, the leaflet support trestle 22 is fabricated from an elastic material that is in compression when attached to the frame base 20. Like the frame base 20, the support trestle 22 can take various shapes and have various cross-sectional geometries. The support trestle 22 can have, e.g., a generally curvilinear (i.e., round or oval) cross-section, or a generally rectilinear cross section (i.e., square or rectangular), or combinations thereof.

The leaflet support trestle 22 can assume various geometric configurations. As shown in FIGS. 4A and 4B, the leaflet support trestle 22 is formed in the shape of an arch.

In FIGS. 4A and 4B, the leaflet support trestle 22 is attached to the frame base 20, e.g., by welding, gluing, or soldering. Other forms of attachment are possible, to accommodate various configurations of the frame base 20, as will be described later.

The leaflet members 16 and 18 are attached to the leaflet support trestle 22. The leaflet support trestle 22 extends from a peripheral region and across and over a midregion of the frame base 20. The trestle 22 extends a vertical distance above the frame base 20, which is dictated by the size of the leaflet members 16 and 18 that are supported by it. In effect, the leaflet support trestle 22 suspends the leaflet members 16 and 18 over the midregion of the frame base 20.

In the embodiment shown in FIGS. 4A and 4B, where both leaflet members 16 and 18 are mobile, the outer edges 24 of leaflet members 16 and 18 are free of attachment to the frame base 20. Thus, the leaflet members 16 and 18 each includes an apex edge 26, along which the leaflet member 16 or 18 is attached to the support trestle 22, and the semi-lunar free edge 24, which is unattached to the frame base 20 and the support trestle 22.

The leaflet members 16 and 18 may comprise natural tissues, elastic shape memory alloys, synthetic polymers and similar biocompatible materials. When mobile, the leaflet member 16 and 18 is desirably pliable. A naturally existing tissue—conventionally chemically fixed by standard available tissue fixatives to prevent shrinkage—may be used as a mobile leaflet member 16 and 18. Alternatively, a mobile leaflet member 16 and 18 may comprise an elastic alloy, like a nitinol membrane, or another pliable synthetic polymer.

A leaflet member 16 and 18 can be attached along its apex edge 26 to the leaflet support trestle 22, e.g., by metal fasteners (as FIGS. 4A and 4B show), or by suture, glue, or any strong bonding agent or element. The attachment desirably occurs all along and on both sides of the apex edge 26.

As shown by arrows in FIG. 4B, the leaflet members 16 and 18 are sized and configured to assume complementing orientations that change according to the direction of blood flow. One complementing orientation (shown in FIG. 4B) intercepts retrograde blood flow, i.e., when blood flows upstream toward the prosthesis 10. The leaflet members 16 and 18 are sized and configured, when incepting the retrograde blood flow, to assume an open cone formation along their semi-lunar free edges 24, as FIG. 4B shows. The conical formation is suspended by the support trestle 22 over the frame base 20. The conical formation extends fully up from the frame base 20 to the apex edges 26 defined by the attachment of the leaflet members to the trestle 22. When the cone formation is opened, the valve path is closed or at least impeded. The open cone formation halts or at least interferes with blood flow in a retrograde direction.

As shown by arrows in FIG. 4A, the leaflet members 16 and 18 are sized and configured to assume a different complementing orientation in response to antegrade blood flow. When blood flows downstream toward the prosthesis 10, the leaflet members 16 and 18 respond by collapsing the cone formation. When the cone formation is collapsed, the valve path is opened. Blood flows along opposite sides of the support trestle 22, in the peripheral channels 28 defined in the spaces that are occupied by mobile leaflet members 16 and 18, when in their cone formation, between the support trestle 22 and the peripheral region of the frame base 20. The collapse of the cone formation permits blood flow in an antegrade direction through the peripheral channels 28.

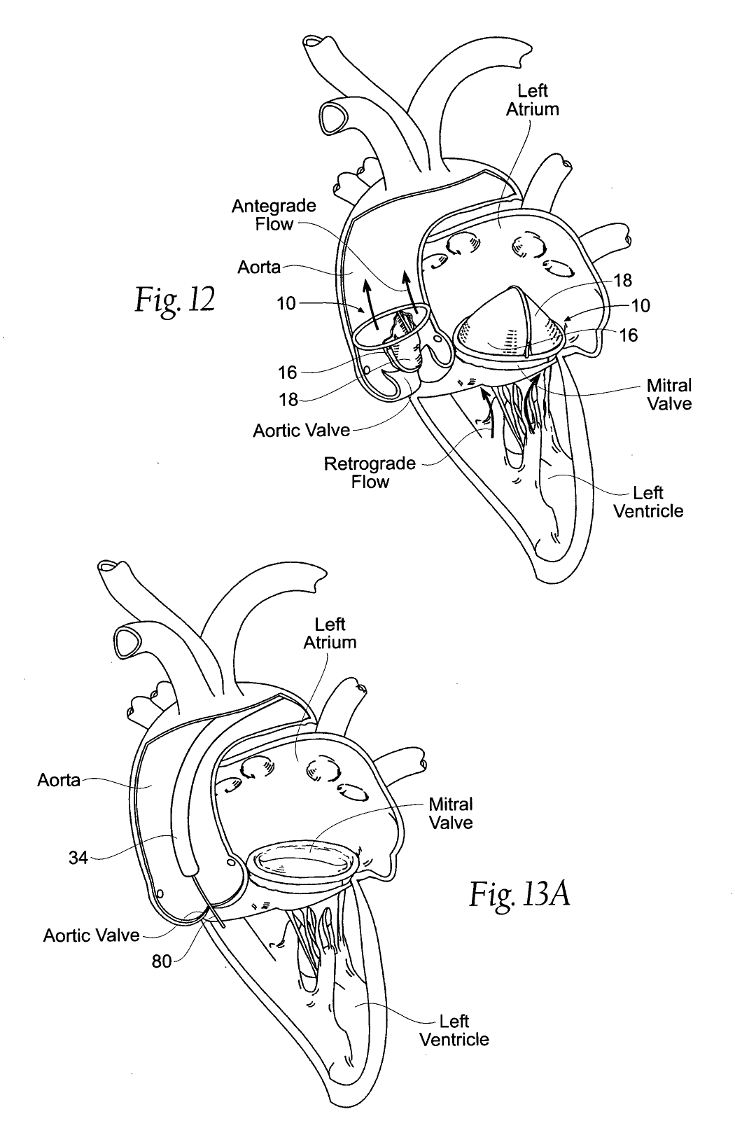

In use (see FIGS. 11 and 12), the prosthesis 10 is implanted in the mid blood stream of a blood path, which can comprise a valve annulus or great vessel. The open end 30 of the prosthesis 10 (i.e., the end that does not include the leaflet support trestle 22) is oriented to face downstream relative to the desired blood flow direction, i.e., so that retrograde blow flow enters the open end 30 of the prosthesis 10. Conversely, the leaflet support trestle 22 is oriented to face upstream relative to the desired flow direction, i.e., so that antegrade flow exits the open end 30 of the prosthesis 10.

Antegrade and retrograde blood flow are driven by the cyclical pumping of blood by the heart, and the particular direction of desired blood flow will vary depending upon the heart valve location. For example, on the left side of the heart, the desired direction of blood flow (antegrade) through the mitral valve is from the left atrium into the left ventricle (see FIG. 11). Conversely, undesired retrograde flow through the mitral valve is from the left ventricle into the left atrium. With respect to the aortic valve, the opposite is true. The desired direction of blood flow (antegrade) through the aortic valve is from the left ventricle into the aorta (see FIG. 12), and undesired retrograde flow through the aortic valve is from the aorta into the left ventricle. The same reverse relationships are also true on the right side of the heart with respect to antegrade/retrograde flow through the tricuspid valve and the pulmonary valve, respectively.

When the open end 30 is properly oriented with respect to the desired direction of blood flow, the mobile leaflet members 16 and 18 respond by assuming different complementing orientations in response to differing hemodynamic pressures, to permit antegrade flow and block retrograde flow.

More particularly, when upstream blood pressure is greater that downstream blood pressure (i.e., the conditions of antegrade flow), the resultant hemodynamic pressure condition pushes against the exterior aspect of the mobile leaflet members 16 and 18. The mobile leaflet members 16 and 18 react by assuming a complementing orientation opening the peripheral flow channels 28 (see FIG. 4A).

When the upstream push subsides and downstream blood pressure becomes greater than upstream pressure (i.e., the conditions of retrograde flow), the resultant hydrodynamic pressure condition pushes against the interior aspect of the mobile leaflet members 16 and 18, central to their conical structure. The mobile leaflet members 16 and 18 react by assuming a different complementing orientation closing the peripheral flow channels 28(see FIG. 4B).

The prosthesis 10 may be attached to a cardiac or vascular tissue region in an open surgical procedure, using sutures passed through a fabric sewing cuff carried by the frame base 20. Adhesives or other fixation materials can be used. Alternatively, or in combination with sutures, adhesives, or other fixation materials, the frame base 20 may include hooks or barbs 32 that penetrate tissue to anchor the prosthesis 10.

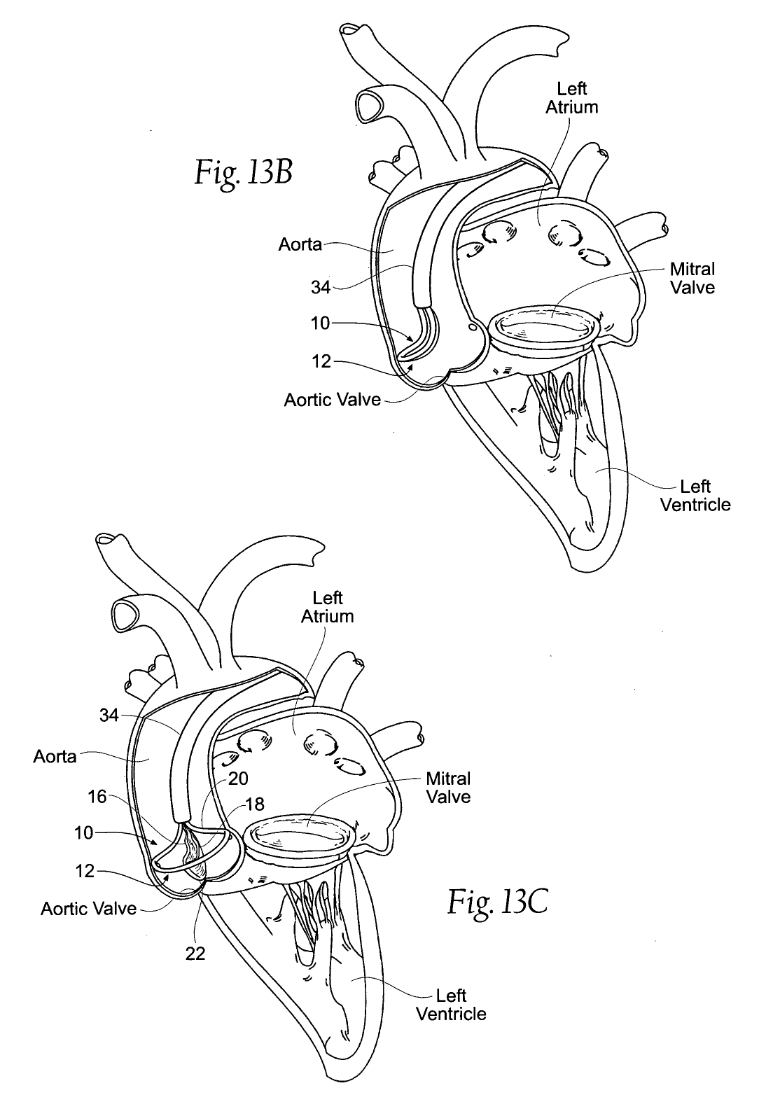

As FIGS. 13A, 13B, and 13C show, an intra-vascular procedure may be used to implant the prosthesis 10. In this arrangement, the prosthesis 10 may be deployed by first folding and/or compressing the frame 12 into a lumen of a transvascular catheter 34 for delivery. The catheter may be advanced through the vasculature into the heart through a retrograde arterial route (as FIG. 13A shows) or an antegrade venous and then trans-septal route, if left heart access is needed from a peripheral vessel access. Use of a standard available guide wire 80 and/or guide sheath will assist the operator in delivering and deploying the catheter 34 into position.

The frame 12 of the prosthesis 10 could then be pushed out of the lumen of the catheter (as FIG. 13B shows). The frame 12 can, e.g., self-expand into the desired shape and tension when released in situ (as FIG. 13C shows). In this arrangement, compression of the frame base 20 against tissue can serve as an attachment force to the native cardiac or great vessel Alternatively, balloon dilation of a malleable frame base 20—or an elastic frame base 20 that at least partially if not fully self-expands upon release—may be used. The frame base 20 may also have hooks or barbs 32 to provide purchase into tissue.

The hoop strength of the leaflet support trestle 22 to which the leaflet members are attached, coupled with the hoop strength and/or barbs 32 of the circumferential mural or annulus frame base 20, desirably serve to anchor the prosthesis 10 in position. In addition (see FIGS. 4A and 4B), the trestle 22 can include ancillary appendages, such as antennae-like, super-elastic tentacles (not shown), that radiate toward the wall of the heart or vessel from the upstream apex of the trestle 22, which can also serve to center and stabilize the prosthesis 10 at its upstream aspect.

Once properly implanted, the prosthesis 10 serves as a peripheral flow suspension valve. The term “peripheral flow” denotes that, unlike a conventional central flow valve, the valve prosthesis 10 functions by allowing blood flow at the periphery of the mobile leaflets members 16 and 18, so that the flow lumen of the valve is outside all mobile leaflet members 16 and 18. Peripheral flow channels 28 are located between a given mobile leaflet member 16 and 18 and a mural wall of a heart, great vessel or native valve annulus. The term “suspension” denotes that, unlike a conventional central flow valve, the leaflet members 16 and 18 are suspended from the trestle above an annulus. The leaflet members 16 and 18 lay aligned in the direction of blood flow, antegrade or retrograde.

Once properly implanted, retrograde blood flow into the open end 30 of the prosthesis 10 fills the interior of the leaflet members 16 and 18 with blood (see FIG. 4B). The leaflet members 16 and 18 thereby halt blood flow in a retrograde direction (see FIG. 4B). This is caused by the mobile leaflet members 16 and 18 moving peripherally at their free edges 24 at the level of the frame base 20 away from the trestle 22 and out toward the peripheral region of the frame base 20. A line of coaptation is formed between the free edges 24 of the mobile leaflet members 16 and 18 and the frame base 20 and/or native tissue circumference where the prosthesis 10 is placed, which can comprise the peripheral wall of the vessel and/or residual native valve annulus or valve tissue. This occurs because retrograde blood flow delivers a push to the central or inner aspect of the mobile leaflet members 16 and 18 suspended at the apex 26 by the trestle 22. By so doing, each mobile leaflet member 16 and 18 contributes partially to the formation of a full cone or cup (that is, by forming complementing orientations), rendering the valve prosthesis 10 competent against retrograde blood flow.

Likewise, the cone conformation collapses in response to antegrade blood flow. The free edges 24 of the mobile leaflet members 16 and 18 move back toward the trestle 22 (see FIG. 4A) to allow blood flow through the channels 28 defined between the outer surface of the mobile leaflet members 16 and 18 and the peripheral region of the frame base 20.

During the cardiac cycle, the free ends 24 of the mobile leaflet members 16 and 18 move cyclically, fanning outward to seal against the frame base 20 and/or native tissue to close the peripheral flow channels 28 and falling back inward to open the peripheral flow channels 28, in response to retrograde and antegrade blood flow, respectively. The valve prosthesis 10 is competent to regulate the direction of blood flow, by allowing a relatively unimpeded forward flow of blood, e.g., toward the aorta in the left heart or pulmonary artery in the right heart, or from the atriums toward the respective left or right ventricle, and by preventing a greater part of a backward flow of blood away from the normal forward flow of blood in one or the other heart cycle, systole or diastole.

FIGS. 5A and 5B show another embodiment of a heart valve prosthesis 36 that embodies features of the invention. Like the previous embodiment, the heart valve prosthesis 36 shown in FIGS. 5A and 5B is sized and configured to supplement, repair, or replace a native heart valve. In FIGS. 5A and 5B, the frame 38 of the prosthesis 36 includes a trestle 44 that supports at least one mobile leaflet member 40, like the mobile leaflet members 16 and 18 as previously described, as well as one immobile leaflet member 42.

The immobile leaflet member 42, like the mobile leaflet member 40, may comprise natural tissue, elastic shape member alloy, synthetic material, or similar biocompatible materials. The immobile leaflet member 42 may be shaped just like a mobile leaflet member 40, except that the immobile leaflet member 42 is fully attached about its periphery to the frame base 46 and the leaflet support trestle 44. That is, the immobile leaflet member 42 has no free edges. Still, the immobile leaflet member 42 is desirably pliable, particularly when intra-vascular delivery is desired. The immobile leaflet member 42 is also firm and turgid with reference to both antegrade and retrograde blood flow. This results in an always-present partial cone formation (see FIG. 5A).

Used in conjunction with at least one mobile leaflet member 40, the immobile leaflet member 42 allows functional closure of the valve as a whole. In this arrangement, the unattached free end 48 of the mobile leaflet member 40 becomes blood filled in response to blood flow in a retrograde direction (see FIG. 5B). This is caused by the mobile leaflet member 40 moving peripherally at its free edge 48 out toward the peripheral region of the frame base 46, where it forms a line of coaptation between the free edge 48 of the mobile leaflet member 40 and the frame base 46 and/or the native tissue circumference where the prosthesis 10 is placed. A transient, partial cone formation results.

The permanent, partial cone formation of the immobile leaflet member 42 complements the transient partial cone formation of the mobile leaflet member 40. Together, the immobile and mobile leaflet members 42 and 40 form a full cone formation or cup, rendering the valve prosthesis 10 competent against retrograde blood flow.

Likewise, the full cone conformation collapses in response to antegrade blood flow, as the free edge 48 of the mobile leaflet member 40 moves back toward the trestle 44 (see FIG. 5A). This allows blood flow in the single peripheral channel So defined between the mobile leaflet member 40 and the peripheral region of the frame base 46. During the cardiac cycle, the free edge 48 of the mobile leaflet member 40 moves cyclically, fanning outward to seal against the peripheral region of the frame base 46 and/or native tissue to close the peripheral flow channel 50 and falling back inward to open the peripheral flow channel 50, in response to retrograde and antegrade blood flow, respectively.

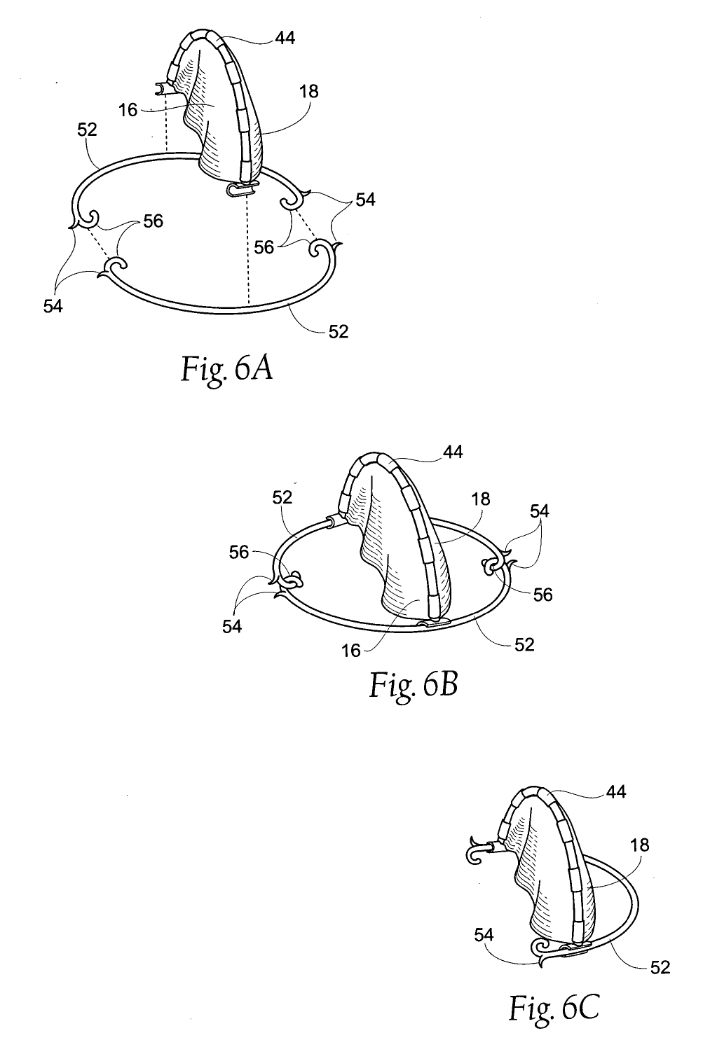

As shown in FIG. 6A, a given frame base 52 can be interrupted to impart a normally open annular shape to the prosthesis 10. The arc defined by the interrupted frame base 52 can, of course, vary. The interrupted frame base 52 can be implanted as is, as FIG. 6B shows. The terminus of an interrupted frame base 52 may include a hook or barb 54 to pierce tissue and anchor the frame base 52 at the preferred position in the heart or great vessel. In this arrangement, anchoring will be dependent upon the hoop strength exerted by the interrupted frame base 52 contacting a vascular wall. Sutures, adhesives, or other forms of attachment can be used to enhance the anchoring.

An interrupted frame base 52 can include interlocking hooks 56 that can be coupled, if desired, to themselves or to another interrupted frame base 52 (see FIG. 6C) to form a composite closed-loop frame. A composite frame need not be completely closed, but could comprise an opened-loop structure as well.

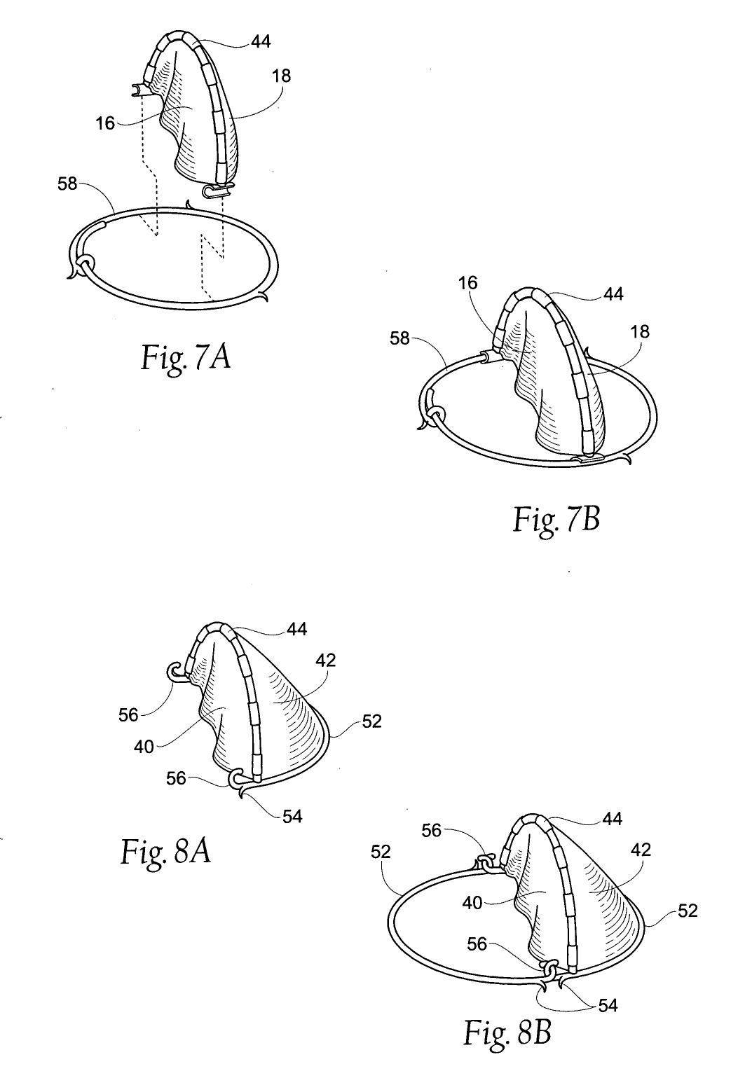

The use of an interrupted frame base 52, or two or more interlocking interrupted frame base 52, provide a degree of adjustability to conform the frame 38 to the native tissue where it is to be attached. A similar degree of flexibility can be achieved by using a sliding frame base 58 structure, as shown in FIG. 7A.

In these arrangements (see FIGS. 6A-C and 7A-B), the leaflet support trestle 44 can comprise a separate component. The separate trestle structure 44 can be clipped or otherwise fitted to an interrupted frame base 52 (as FIG. 6B shows) or across interlocked frame bases 52 (as FIG. 6C shows), or across a sliding frame base structure 58 (as FIGS. 7A and 7B show).

In FIGS. 6A-C and 7A-B, the leaflet support trestle 44 carries two mobile leaflet members 16 and 18. As FIGS. 8A and 8B show, an interrupted frame base 52 can carry a leaflet support trestle 44 having an immobile leaflet member 42, which is attached to the interrupted frame base 52, in association with a mobile leaflet member 40, which is free of attachment to the interrupted frame base 52. The interrupted frame base 52 can be implanted as an open-loop, as shown in FIG. 8A. Alternatively, as FIG. 8B shows, an interrupted frame base 52 carrying the immobile and mobile leaflet members 42 and 44 can be interlocked with another interrupted frame base 52 that carries no leaflet members (e.g., using the interlocking hooks 56), to form a close-loop composite frame.

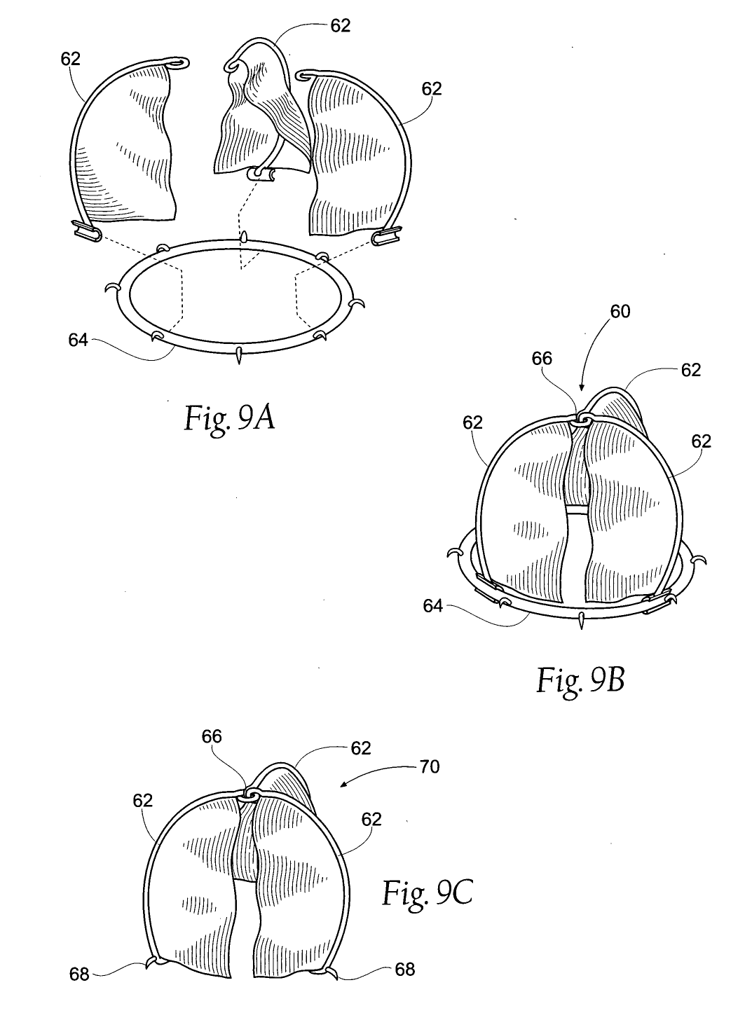

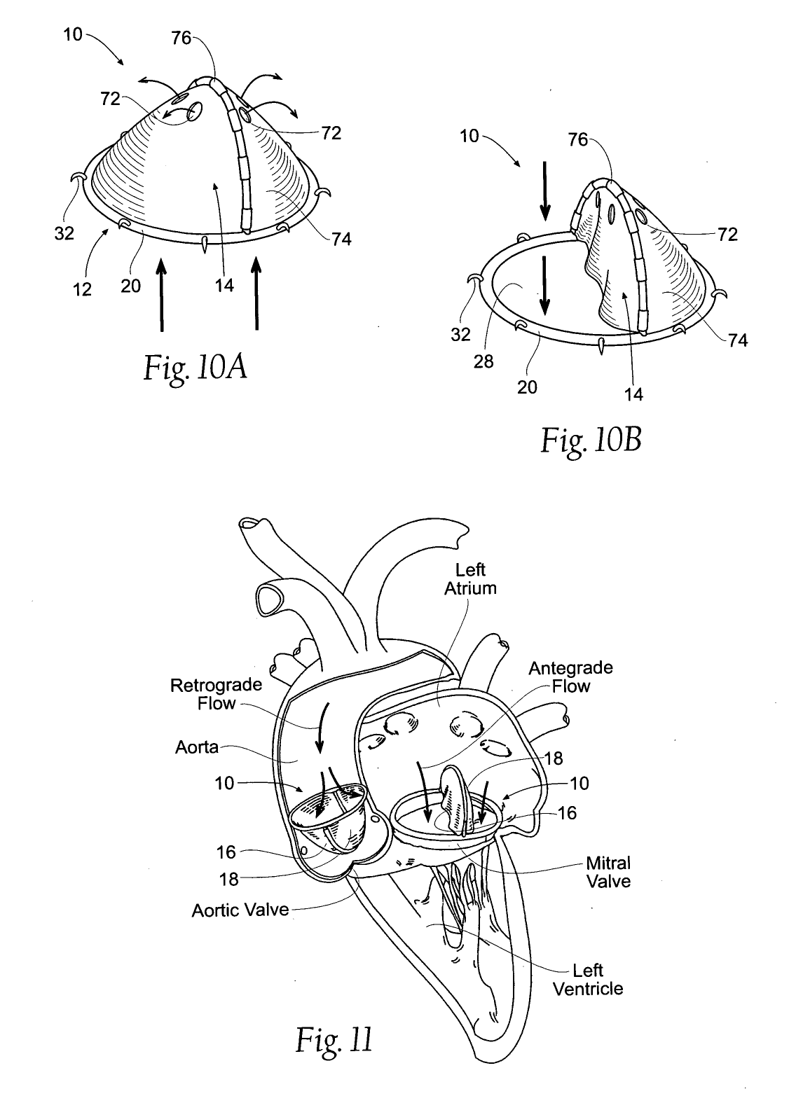

In the preceding embodiments, a single trestle 22 or 44 is used, which spans across and above the center region of the valve prosthesis 10. This structure accommodates the use of two leaflet members, comprising either two mobile leaflet members 16 and 18 or a mobile leaflet member 40 used in conjunction with an immobile leaflet member 42. As FIGS. 9A and 9B show, a composite trestle 60 can be constructed using an assembly of three trestle members 62 that are coupled 120-degrees apart about the frame base 64 and joined at a common apex 66. The three trestle members 64 form a tripod-like composite trestle 60 braced by the apex 66 in the center of the frame base 64.

The tripod-like composite trestle 60 makes possible a tri-leaflet valve function comprising three mobile leaflet members, or at least one mobile leaflet member in combination with at least one immobile leaflet members, or permutations thereof.

As FIG. 9C shows, each trestle member 62 may include an anchoring hook 68 that individually anchors the trestle member 62 into a heart annulus, heart valve tissue, or a vessel wall. Anchored circumferentially in tissue and joined at the apex 66, a tripod-like leaflet suspension prosthesis 70 can be created, which can be implanted and stabilized against migration within the heart or in a greater vessel without the use of a peripheral frame base. The elimination of a peripheral frame base simplifies delivery and implantation, particularly by intra-vascular, catheter-based techniques.

In FIGS. 10A and 10B, one or more gaps 72 or holes can be formed in at least one leaflet member 74 or in the attachment between a leaflet member 74 and a trestle 76. The gap or gaps 72 are desirably proximate to the apex 78 of the trestle 76, or can be appear intermittently along points of attachment to the trestle 76. The gap or gaps 72 pass blood to wash the inner and/or central surface of the leaflet member or members 74, allowing a degree of back flow of blood to leak through the gap or gaps 72 when the leaflet members 74 are otherwise closed to retrograde backflow of blood. A backflow of blood through the gaps 72 make possible a circulation of blood to wash the inner surfaces of the leaflet members 74, to decrease the possibility of thrombus formation within the leaflet assembly 14. Gap or gaps 72 may be provided in either mobile or immobile leaflet members, as FIGS. 10A and 10B show.

FIGS. 11 and 12 show a bileaflet valve prosthesis 10 of the type shown in FIGS. 4A-B implanted in a heart (left atrium) and a great vessel (aorta). One bileaflet valve prosthesis 10 is implanted in the aorta immediately superior to the aortic valve. Another bileaflet valve prosthesis 10 is implanted in the left atrium immediately superior to the mitral valve.

In the aortic valve position, the valve prosthesis 10 could be deployed through the aorta retrograde up a peripheral artery. Alternatively, it may be passed from a peripheral vein through the atrial septum across the mitral valve and into position somewhere in the left ventricular to aortic outflow tract. In the mitral valve position, either approach trans-arterial and trans-aortic, or trans-venous and then trans-septal, could be done.

In the aortic valve location, the prosthesis 10 is placed at or in the leaflets of the native aortic valve. The native leaflets may be left intact, and the base of the prosthesis 10 pressed up against them, or a hook on the base of the prosthesis 10 may be hooked into the annulus or into the aorta well above the coronary arteries. The native leaflets could be left there, if they are flimsy as in the case of aortic regurgitation like that due to annular dilation like Marian's. If the native leaflets are calcified, they may be stretched open by a stretcher device that could be advanced intravascularly. In certain cases it may be possible to remove the calcium from the leaflets. Or, the calcified leaflets may be left behind and propped open by the frame base 20 of the prosthesis 10.

In the mitral valve location, the prosthesis 10 is placed at or in the leaflets of the native mitral valve. The native leaflets may be left intact, and the base of the prosthesis 10 pressed up against them, or a hook on the base of the prosthesis 10 may be hooked into the annulus or into the left atrial wall above the mitral valve.

During ventricular diastole (when the left ventricle fills) (see FIG. 11), blood rushes back retrograde from the aorta toward the left ventricle. The two leaflet members 16 and 18 of the prosthesis 10 in the aortic valve location fill like an umbrella up against the native aortic valve annulus at the base of the prosthesis 10. The retrograde flow of blood from the aorta into the left ventricle from the aorta is blocked. If present, the calcified native leaflets cannot close because they are stiff, so the leaflet members 16 and 18 fill and close off blood flow in diastole by contacting the remnant aortic leaflets or aortic wall in the aorta.

Concurrently, during ventricular diastole, the two leaflet members 16 and 18 of the prosthesis 10 in the mitral valve location collapse to permit antegrade flow from the left atrium into the left ventricle.

During ventricular systole (when the left ventricle empties) (see FIG. 12), the blood rushes back retrograde from the left ventricle toward the left atrium. The two leaflet members 16 and 18 of the prosthesis 10 in the mitral valve location fill like an umbrella, blocking retrograde flow from the left ventricle into the left atrium. Concurrently, the two leaflet members 16 and 18 of the prosthesis 10 in the aortic valve location collapse to permit antegrade flow of blow from the left ventricle into the aorta.

While the new devices and methods have been more specifically described in the context of the treatment of a mitral heart valve or an aortic heart valve, it should be understood that other heart valve types can be treated in the same or equivalent fashion. By way of example, and not by limitation, the present systems and methods could be used to prevent or resist retrograde flow in any heart valve annulus, including the tricuspid valve, the pulmonary valve, as well as the aortic valve and the mitral valve. In addition, other embodiments and uses of the invention will be apparent to those skilled in the art from consideration of the specification and practice of the invention disclosed herein. The specification and examples should be considered exemplary and merely descriptive of key technical features and principles, and are not meant to be limiting. The true scope and spirit of the invention are defined by the following claims. As will be easily understood by those of ordinary skill in the art, variations and modifications of each of the disclosed embodiments can be easily made within the scope of this invention as defined by the following claims.

1. A valve prosthesis comprising a frame sized and configured to rest within a blood path subject to antegrade and retrograde blood flow, the frame including a trestle element that extends across the blood path and a leaflet assembly suspended from the trestle element that extends into the blood path in alignment with blood flow, the leaflet assembly including at least one mobile leaflet member sized and configured to assume orientations that change according to blood flow direction, the mobile leaflet member having an first orientation that permits antegrade blood flow and a second orientation that resists retrograde blood flow.

2. A valve prosthesis according to claim 1
wherein the leaflet assembly includes at least one immobile leaflet member sized and configured with a fixed orientation that resists retrograde blood flow, the second orientation of the mobile leaflet member complementing the fixed orientation of the immobile leaflet member during retrograde flow.

3. A valve prosthesis according to claim 2
wherein the fixed orientation and second orientation define a cone having an apex at the trestle element.

4. A valve prosthesis according to claim 1
wherein the leaflet assembly includes at least first and second mobile leaflet members sized and configured to assume complementing orientations that change according to blood flow direction, the first and second mobile leaflet members having a first complementing orientation that permits antegrade blood flow and a second complementing orientation that resists retrograde blood flow.

5. A valve prosthesis according to claim 4
wherein the complementing second orientations define a cone having an apex at the trestle element.

6. A valve prosthesis according to claim 1
wherein the leaflet assembly includes at least first, second, and third leaflet members, at least one of the leaflet members comprising the mobile leaflet member, the first, second, and third leaflet members having a first complementing orientation that permits antegrade blood flow and a second complementing orientation that resists retrograde blood flow.

7. A valve prosthesis according to claim 1
wherein the frame includes a peripheral region and a midregion, and
wherein the trestle element spans from the peripheral region across and above the midregion.

8. A valve prosthesis according to claim 7
wherein, when the mobile leaflet member is in the first orientation, a through-flow path is defined between the trestle element and the peripheral region.

9. A valve prosthesis according to claim 8
wherein, when the mobile leaflet member is in the second orientation, the mobile leaflet member extends from the trestle element toward the peripheral region to close the through-flow path.

10. A valve prosthesis according to claim 1
wherein the frame includes an elastic structure.

11. A valve prosthesis according to claim 10
wherein the elastic structure is collapsible for placement within a catheter.

12. A valve prosthesis according to claim 10
wherein the elastic structure includes a spring-memory material.

13. A method of supplementing, repairing, or replacing a native heart valve comprising implanting a valve prosthesis as defined in claim 1.

14. A method according to claim 13, wherein the valve prosthesis is implanted by an intra-vascular approach.
